(19) United States
(12) Patent Application Publication
(10) Pub. No.: US 2005/0136538 A1
(43) Pub. Date: Jun. 23, 2005

(54) Lithographic method for attaching biological cells to a solid substrate using a small molecule linker

(76) Inventors: Srikant Pathak, Pleasanton, CA (US); Blake Simmons, San Francisco, CA (US); Paul M. Dentinger, Sunol, CA (US)

Correspondence Address: Timothy Evans;Sandia National Laboratories, MS 9031, 7011 East Avenve, Livermore, CA, 94550, US

(21) Appl. No.: 10/739,493

(22) Filed: Dec. 17, 2003

(51) Int. Cl. C12N 5/00; C12N 5/02

(52) U.S. Cl. 435/395; 435/396; 435/402; 514/2

(57) ABSTRACT

One embodiment of the invention describes a novel method for providing a substrate for selective cell patterning, wherein the method comprises contacting an epoxide coated substrate surface with a bi-functional molecule having an epoxide end group and reacting these end groups with the substrate surface through a photochemically induced acid coupling reaction. The bi-functional molecule is applied in solution to the substrate surface as a photo-sensitive coating. A photomask stencil is used to deposit electromagnetic radiation into the coating in predetermined locations to form a desired pattern of the coating. The patterned substrate is provided by washing coating from the substrate leaving the bi-functional molecule attached to the substrate in those areas exposed to the radiation providing thereby cell adhesive moieties in controlled locations on the substrate.

The invention described below was made with Government support under government contract no. DE-AC04-94AL85000 awarded by the U.S. Department of Energy to Sandia Corporation. The Government has certain rights in the invention, including a paid-up license and the right, in limited circumstances, to require the owner of any patent issuing in this invention to license others on reasonable terms.

The subject matter of the present technical disclosure relates to a method for attaching biological matter such as cells or cell fragments to a solid substrate. More particularly, an embodiment of the invention relates to the non-intuitive use of a small hydrophobic molecule to attach cells to a solid substrate.

The ability to pattern cells and other biological matter is desirable from many perspectives. New scientific and security needs have hastened effort in the development of sensitive sensors for detection of biological agents. At the same time, growing interest in bio-based materials has prompted the use of DNA, proteins and cells as a functional building block. The ability to specifically pattern cells in particularly on inorganic substrates with high structural integrity and viability is a major challenge to be overcome, if cells are to be manipulated in ways similar to other biological entities, (see for instance M. Yousaf, et al., Angew. Chem. Int. Ed. 2001, 40, 1093-1096; X. Jiang, et al., J. Am. Chem. Soc. 2003, 125, 2366-2367; P. Ghosh, et al.; Angew. Chem. Int. Ed. 1999, 38, 1592-1595; A. Rezania, et al., Langmuir 1999, 15, 6931-6939; D. Nicolau, et al., Biosensors & Bioelectronics 1999, 14, 317-325; P. St John, et al., J. Neurosci. Methods 1997, 75, 171-177; and D. Chiu, et al., Proc. Natl. Acad. Sci. USA, 2000, 97, 2408-2413). Furthermore, in the past decade, a number of cell-patterning approaches have been shown using biocompatible polymers, proteins, peptides and small molecules (A. Razatos, et al., Proc. Natl. Acad. Sci. USA, 1998 95, 11059-11064; G. Burks, et al., Langmuir 2003, 19, 2366-2371; Y. Fang, et al., Langmuir, 2003, 19, 1500-1505; C. Nelson, et al., Langmuir 2003, 19, 1493-1499; and E. Endler, et al., Biotech. Bioengg. 2003, 81, 719-724). While some degree of specificity has been achieved, most approaches use multilayer coatings of polymers/small molecules where the role of individual components to cellular adhesion is not clearly understood.

In particular, the study of cell activity while immobilized and the observation of cell growth deal with studying disease in healthy cells or regeneration of tissues. Also of current interest is the detection and identification of biological agents; that is, to recognize cells of specific organisms. We are presently investigating this latter aspect by attempting to provide a practical means for immobilizing cells on a flat surface in order that they might be probed using an optical (fluorescence) and/or an electrical (impedance) method. However, key to the realization of these efforts is the ability to reliably anchor/immobilize cells on a surface.

Methods have been described for making micro-arrays of a single cell type on a common substrate for other applications. One example is a photochemical resist-photolithography technique described by Mrksich and Whitesides, (Ann. Rev. Biophys. Biomol. Struct. 25:55-78, 1996), in which a glass plate is uniformly coated with a photoresist and then “patterned” using a transmission or reflectance mask in a photolithographic process to define the desired “array”. Upon exposure to light, the photoresist in the unmasked areas is removed. The entire photolithographically defined surface is uniformly coated with a hydrophobic substance such as an organosilane that binds both to the areas of exposed glass and the areas covered with the photoresist. The photoresist is then stripped from the glass surface, exposing an array of spots of exposed glass. The glass plate is then washed with an organosilane solution having terminal hydrophilic groups or chemically reactable groups such as amino groups. The hydrophilic organosilane binds to the spots of exposed glass with the resulting glass plate having an array of hydrophilic or reactable spots (located in the areas of the original photoresist) across a hydrophobic surface. The array of spots of hydrophilic groups provides a substrate for non-specific and non-covalent binding of certain cells.

Another example recites a method wherein stamping is used to provide a surface coated with protein adsorptive alkanethiol. (U.S. Pat. No. 5,776,748). In this example a bare gold surface is coated with polyethylene-glycol-terminated alkanethiols that resist protein adsorption. After exposure of the entire surface to laminin, a cell-binding protein found in the extracellular matrix, living hepatocytes attach uniformly to, and grow upon, the laminin coated islands.

These and other methods are therefore generally illustrative of the current state of the art. We have found, however, a non-intuitive process for providing micro-arrays of biological matter, including single cell types, wherein the biological material is preferentially attached to a hydrophobic specie patterned on a surface.

Therefore, one embodiment of the Applicants' invention relates to a simple method for immobilizing cells and other biological material on a flat surface and for detecting the presence of this material using an optical (fluorescence) or electrical (impedance) method.

Another embodiment relates to reliably anchoring/immobilizing cells on a surface by using a small intermediate bi-functional molecule acting as a “negative” resist layer.

Yet another embodiment relates to forming a patterned image of glycidyl 4-nonylphenyl ether (“GNPE”) onto a surface of a silicon wafer modified to include an epoxy-siloxane surface layer.

These and other embodiments of the invention will become apparent to those of skill in this art on reading the invention as it is described and claimed in detail below and as illustrated in the following drawings, briefly described.

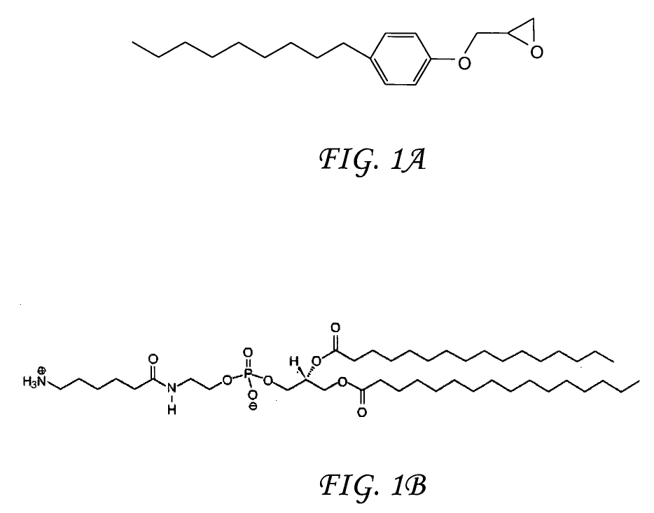

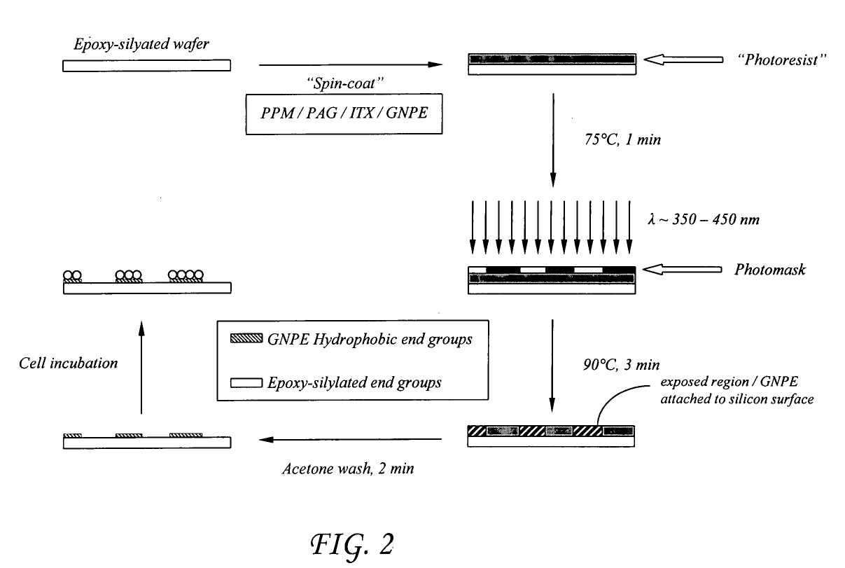

FIG. 1A illustrates a “stick” diagram of the structure of GNPE (glycidyl 4-nonylphenyl ether).

FIG. 1B illustrates a “stick” diagram of the structure of an aminated phospholipid.

FIG. 2 illustrates the lithographic process used to apply an exposed image onto the surface of a silicon wafer.

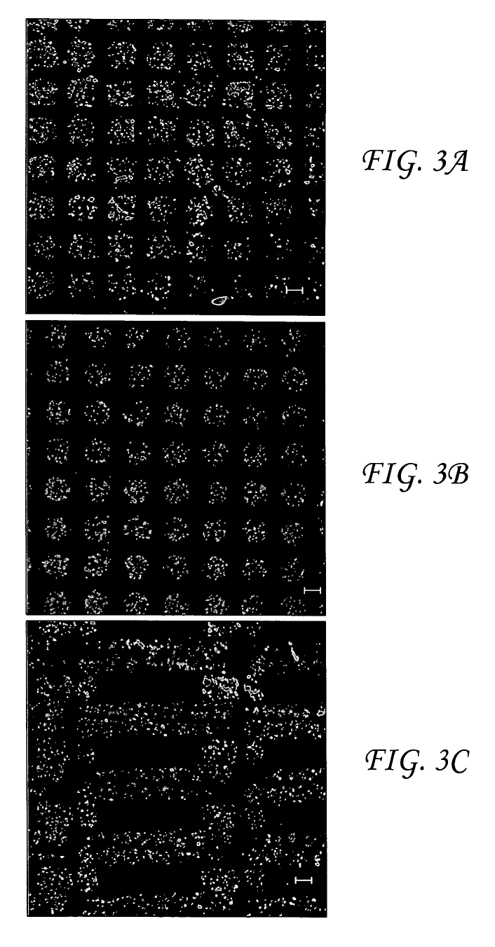

FIGS. 3A-C show the resulting patterns wherein cells of E. coli (FIGS. 3A, C) and cells of B. subtilis (FIG. 3B) are immobilized on a patterned GNPE modified surface and stained with a fluorescent nucleic acid stain.

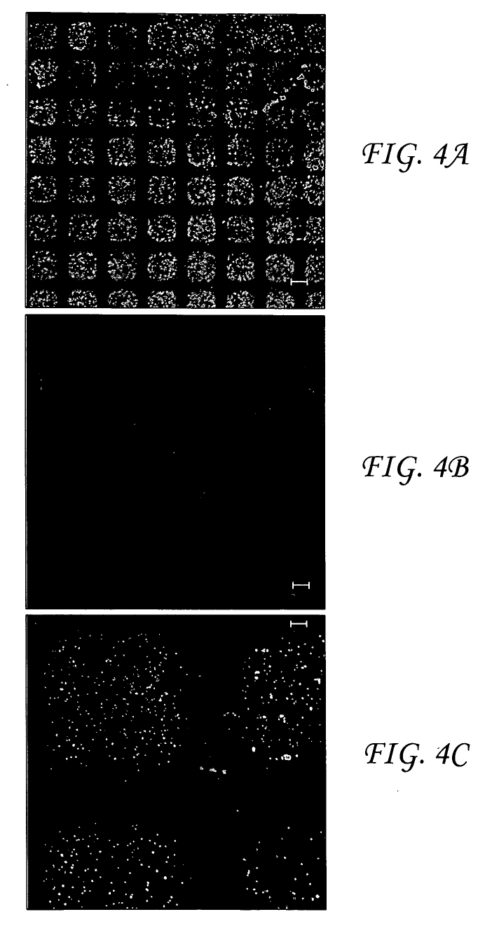

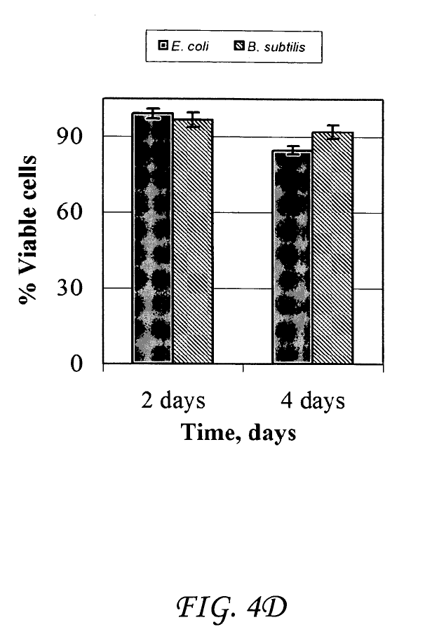

FIG. 4A shows E. coli cells immobilized on a patterned GNPE modified surface and stained, that are not viable (light areas).

FIG. 4B shows E. coli cells immobilized on a patterned GNPE modified surface and stained, that are viable (light areas).

FIG. 4C shows a 1000× view of the patterned GNPE modified surface showing both live and dead E. coli cells.

FIG. 4D illustrates the viability persistence of E. coli cells immobilized on the patterned GNPE modified surface at 2 and 4 days, respectively.

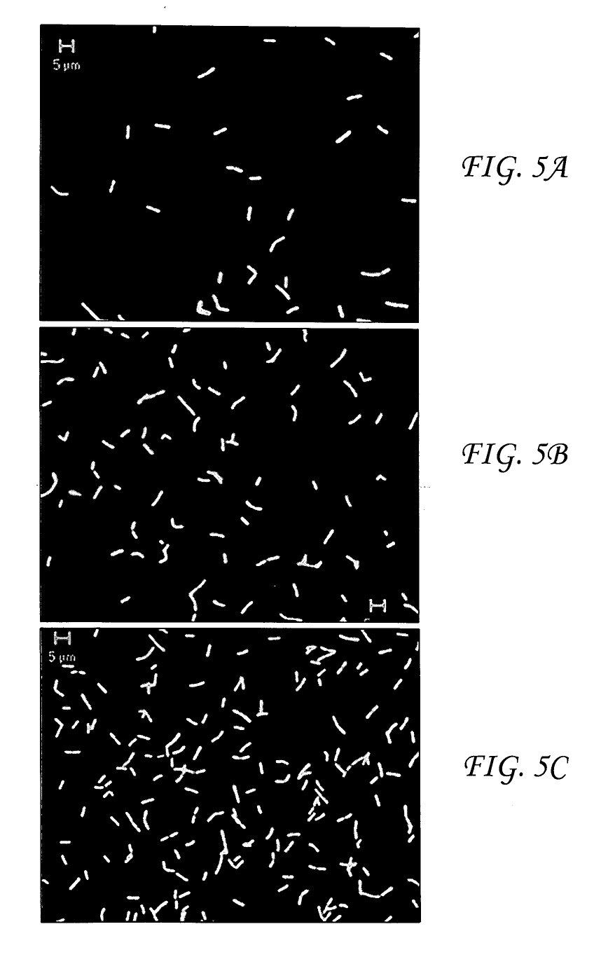

FIG. 5A-C show E. coli incubated on generation-2, -3, and -5 dendrimers mediated GNPE modified surfaces, respectively.

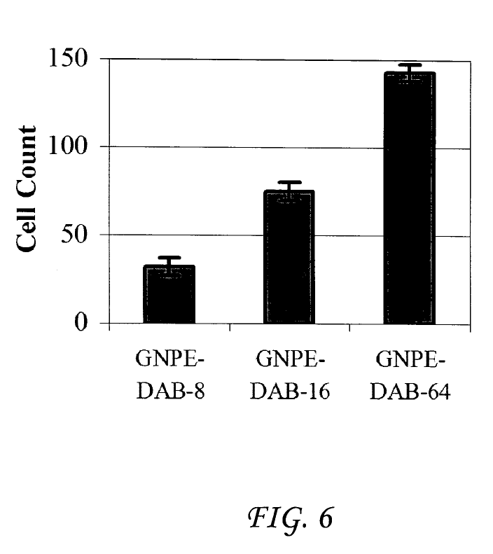

FIG. 6 shows the increase in cell population when E. coli is immobilized under identical conditions on generation-2, -3, and -5 dendrimers mediated GNPE modified surfaces of identical area.

In one embodiment of the invention we have demonstrated preparing a patterned surface for cell immobilization using a recently developed method for patterning oligonucleotides on silicon. In particular, it has been has been discovered that glycidyl 4-nonylphenyl ether (“GNPE”), can be attached to an epoxy-siloxane modified surface of a silicon wafer through an acid catalyzed coupling between the epoxide groups present on the modified silicon surface and the GNPE (see FIG. 1). Moreover, when disposed in this manner, GNPE will act to create a generally hydrophobic layer on the modified silicon surface.

In order to attach the GNPE to the epoxide coated silicon surface, a “resist” layer is applied to that surface which comprises a photoacid generator and a photo absorber sensitive to a specific range of light wavelengths. When the resist layer is exposed to light with these wavelengths, an acid species is formed through a photochemical reaction between a photoacid generator and a photo absorber. The acid species then promotes a coupling reaction between the GNPE and the epoxide groups of the modified surface layer binding the GNPE to the coated surface of the wafer. It was later discovered that biologically active cells would preferentially bind to the exposed hydrophobic portion of the GNPE molecules attached to modified silicon surface.

As seen in FIG. 1, the structure of GNPE exhibits both an epoxide and an alkyl terminal group. GNPE is thus characterized herein as “bi-functional” since it manifests two functional units having utility in the context of the present disclosure.

The process for preparing a silicon substrate for patterning is accomplished in three broad steps. The wafer is first modified by coating it with an epoxide-siloxane monolayer. The then modified wafer is coated with a photoresist-like polymer mixture containing the GNPE, and the “resist” layer is then exposed to specific wavelengths of light energy after which the “resist” is washed away. A detailed description of the materials, the preparation of the polymer mixture and the two step process follows below.

In particular, polished, semi-standard wafers (purchased from International Wafer Services (Portola Valley, Calif.) were first cleaned by rinsing them in NanoStrip® cleaner (obtained from Rockwood Specialties Inc., Princeton, N.J.) for 15 minutes at 60° C. followed by thoroughly rinsing them in deionized (“DI”) water and drying under a flow of dry argon. The silicon wafers were modified by applying a liquid layer of a 1.5% solution of glycidyl oxypropyl trimethoxy silane and acetic acid catalyst in p-xylene to a surface of the wafer. The layer is then reacted with the silicon surface of the wafer to form an epoxy-siloxane monolayer by heating the coated wafer to about 100° C. for 2 hrs., dropping the temperature to about 80° C. for an additional 2 hrs., and then allowing the coated wafer to cool down to room temperature. Wafers modified in this manner were then washed thoroughly with ethanol to remove unreacted silane and unbonded siloxane from the surface.

A photo-reactive polymer solution comprising poly(methyl methacrylate) (“PMMA”) having a molecular weight of about 495 kDaltons (obtained from Microlithography Chemical Corp., Newton, Mass.), 4-octyloxyphenyl phenyliodonium hexafluoroantimonate (“UV 9392C,” obtained from the GE Specialty Materials, Silicones Division, Waterford, N.Y.), isopropyl-9H-thioxanthen-9-one (“ITX,” obtained from the Sigma-Aldrich Company, St. Louis, Mo.) and glycidyl 4-nonylphenyl ether (“GNPE,” obtained from the Sigma-Aldrich Company, St. Louis, Mo.) was prepared in chlorobenzene. In this formulation, PMMA acts as a carrier media, UV9392C is a photoacid generator, and ITX acts as a photo-absorber sensitive to radiation under wavelengths of about 450 nm. Other wavelength ranges are, of course, possible, depending on the desired range and the photo-absorber used.

The photolithographic process shown and described in FIG. 2 comprises spin casting the photo-reactive solution onto the modified silicon wafer for 1 min at 3000 rpm, followed by soft baking at 75° C. for 1 min. The wafer was then exposed using a photomask in broadband UV light with a wavelength range of about 300 nm to about 450 nm in a Karl Suss lamp aligner (model MA6/BA6) to provide an exposure dose of about 550 mJ/cm². The energy of the radiation received by the resist layer is absorbed by the photo-absorber which in turn transfers that energy to the photo-acid generator which generates an acid specie.

After exposure, the wafer was baked at 90° C. for 3 minutes to accelerate a reaction between the acid specie, the GNPE, and the epoxy-siloxane monolayer in those areas exposed to the light radiation in order to covalently bond the GNPE to the epoxy modified silicon surface. Following the baking step the resist material was removed using an acetone wash.

Cell strains of Escherichia coli (“E. coli”), a gram negative bacteria, and Bacillus subtilis (“B. subtilis”), a gram positive bacteria, cells were obtained from the American Type Culture Collection (“ATCC,” Manassas, Va.). Cultures of E. coli K-12 (ATCC strain 29181) and B. subtilis (ATCC strain 13597) were grown overnight at 37° C. in an incubator to achieve saturation conditions. A 1:10 volumetric dilution of the cell culture was then allowed to grow in the Luria Bertani (“LB”) broth into the late log phase to a cell concentration of 6×10⁸ cells/mL. Cells were centrifuged at 5000 rpm for 10 minutes to remove the broth and were re-suspended in deionized water (DI) to a desired final volume to reach an appropriate cell concentration, typically about 10⁵ cells/mL. The mean diameter of dispersed E. Coli, using a light-scattering technique, was found to be 1.287 μm.

Cells produced in this way were then placed onto the pre-patterned substrates via transfer pipette and incubated at 4° C. for 48 hours. For analysis, immobilized cells were stained either with (a) SYTO® 9 green-fluorescent nucleic acid stain or (b) SYTO® 9-propidium iodide live/dead BAClight® bacterial stain (both obtained from Molecular Probes, Inc., Eugene, Oreg.), by incubating the respective solutions with the cell-immobilized surface for 15 minutes. The substrates were then washed thoroughly with DI water or with 1% Triton X-100 or 2% Tween 20 followed by a DI water rinse and then imaged with a model LSM 5 Pascal laser scanning microscope (Carl Zeiss, Inc. New York, N.Y.).

FIGS. 3A-C show the resulting patterns with E. coli (FIGS. 3A, C) and B. subtilis (FIG. 3B) cells specifically immobilized on a patterned GNPE modified surface and stained with SYTO® 9 nucleic acid stain. Very high specificity with respect to cell binding was observed on exposed (squares, circles) areas with GNPE. Very low binding was observed on the unexposed areas of the surface. The process is highly reproducible, with very similar patterning efficiency found across the patterned surface, typically an area of 2.5 cm².

FIGS. 4A-C show the viability studies of the immobilized cells using live-dead assays. Cells that are not viable appear red (FIG. 4A, emission at 635 nm) while living cells appear green (FIG. 4B, emission at 500 nm) under excitation at 480 nm. FIG. 4C shows a composite red-green image of both live and dead cells at 1000×. Moreover, viability studies of patterned cells indicated over 85% of both E. coli and B. subtilis cells remained viable after 4 days of incubation on the surface (FIG. 4D).

To test the assertion that GNPE is responsible for such highly specific patterning, surfaces with different relative amounts of GNPE were prepared. For that, polypropylenimine dendrimer (generation-2, generation-3 and generation-5) modified surfaces were prepared by activating the native hydroxyl groups of cleaned silicon surface first with 1, 1′-carbonyldiimidazole and then reacting with the respective dendrimer solution at room temperature for 12 hrs. To create the GNPE modified surface, a mixture of GNPE in methanol was further reacted with the dendrimer modified surface overnight at room temperature. The surface was rendered very hydrophobic once GNPE modification was completed. Contact angle measurement with water showed an angle of 70°-75° for various dendrimers mediated GNPE modified surfaces. The difference in contact angle between generation-1, generation-2 and generation-5 was 5°-7°, which is within the limit of our measurement capability.

As seen in FIGS. 5A-C, respectively, much different cell coverage was observed on each of these three substrates after E. coli was incubated with these surfaces. In particular, FIG. 6 shows that the cell coverage in the case of generation-5 dendrimer-GNPE (FIG. 5C), was over 3 times the coverage in case of generation-2 dendrimer-GNPE surface (FIG. 5A). Moreover, the accessible amine content of the dendrimer modified surfaces was found to be about 4 times greater in the case of generation-5 than in the case of generation-2. The increase in E. coli density with increasing concentration of GNPE in each subsequent generation clearly suggests its role in cell immobilization. It seems plausible that the GNPE tail, which is structurally similar to the hydrophobic end groups of molecules such as cholesterols and phospholipids found in the cell membrane, is interacting with the cell membrane.

Under distress conditions (alkalinity, nutrient deficiency, etc.) cells are known to produce hydrophobic molecules such as long chain phospholipids that render the cell surface more hydrophobic. As a result, cells grown under rich nutrient conditions (such as in an LB solution) tend to be less hydrophobic than the cells grown under poor nutrient conditions (such as in PBS 7.4 buffer or in D.I. water). Non-specific attachment and poor patterns were observed when cell immobilization on the patterned substrate was performed right after the LB growth phase as compared to when the cells were allowed to further incubate in D.I. water or PBS 7.4 for at least 48 hrs. This result, in tandem with the GNPE concentration dependent cell coverage suggests that the interaction between GNPE and hydrophobic membrane components (such as phospholipids) present in the cell membrane is most likely the basis of highly specific cell patterns. Moreover, the high viability of immobilized cells also indicate that this interaction is not necessarily detrimental to the cells.

In summary, the foregoing disclosure provides an account of a simple method for patterning bacterial cells. In particular, direct patterning of cells has been described using a hydrophobic small molecule, such as GNPE. Very high viability rates were obtained for cells immobilized using this approach. The role of the underlying hydrophobic molecule (GNPE) was ascertained by changing its density, growth conditions and by cell-viability studies. The method described is a fairly general and versatile process that has been shown to work both with a gram positive (B. subtilis) and a gram negative (E. coli) bacteria. Approaches like this, using a specific small molecule pattern, when used in tandem with lipid, sugar, and protein arrays should further enhance our understanding of the molecular basis of cellular attachment and signaling processes. Because of the inherent ease and highly facile process and because molecules like GNPE are structurally very similar to phospholipids, it is expected that this method will find utility in the area of biodetection, biosensors and membrane transport studies.

1. A method for attaching a biological cell or cell constituent to a solid substrate, comprising the steps of:
a.) attaching a bi-functional molecule comprising a short chain alkyl to the solid substrate; and
b.) attaching the biological cell or cell constituent to the short chain alkyl.

2. The method of claim 1, wherein said solid substrate comprises an epoxide layer.

3. The method of claim 2, wherein said bi-functional molecule is attached to the epoxide layer.

4. The method of claim 1, wherein said bi-functional comprises glycidyl 4-nonylphenyl ether.

5. A method for attaching a biological cell or cell constituent to a solid substrate, comprising the steps of:
a.) forming an epoxy-siloxane monolayer on a surface of a silicon wafer;
b.) coating said surface with a solution comprising glycidyl 4-nonylphenyl ether;
c.) forming a pattern on said surface by covalently bonding a portion of said glycidyl 4-nonylphenyl ether to predetermined areas on said epoxy-siloxane monolayer;
d.) preparing a concentrated aqueous suspension of biological cells;
e.) applying a portion of said aqueous suspension to said predetermined areas; and
f.) incubating said silicon wafer and said cell suspension for a predetermined time and temperature.

6. The method of claim 5, wherein the step of forming an epoxy-siloxane monolayer further comprising the steps of:
a.) cleaning and drying said surface of said silicon wafer;
b.) applying an epoxy-silane polymer solution to said surface;
c.) reacting said silicon wafer and said epoxy-silane polymer solution to provide an epoxy-siloxane monolayer covering said surface; and
d.) washing said epoxy-silane monolayer to remove unreacted silane and unbonded siloxane.

7. The method of claim 6, wherein the step of applying the epoxy-siloxane monolayer further comprises the steps of:
a.) forming a 1.5% resist solution comprising glycidyl oxypropyl trimethoxy silane and acetic acid catalyst in p-xylene; and
b.) coating said surface with said resist solution to form a thin liquid layer.

8. The method of claim 6, wherein the step of heating further comprises the steps of:
a.) heating the coated wafer to about 100° C. for 2 hrs.,
b.) decreasing the temperature to about 80° C. for an additional 2 hrs.; and
c.) cooling the coated wafer to room temperature.

9. The method of claim 5, wherein the step of coating said surface further comprises the steps of:
a.) forming a photo-reactive polymer solution comprising an organic solvent, a polymer carrier media, a photoacid generator, a photo-absorber sensitive to a predetermined range of electromagnetic wavelengths, and said glycidyl 4-nonylphenyl ether;
b.) applying said photo-reactive polymer solution to said surface to provide a thin liquid coating; and
c.) drying said liquid coating to provide a photo-reactive layer.

10. The method of claim 9, wherein said carrier media is poly(methyl methacrylate), said photoacid generator is 4-octyloxyphenyl phenyliodonium hexafluoroantimonate, said photo absorber is isopropyl-9H-thioxanthen-9-one, and said organic solvent is chlorobenzene.

11. The method of claim 9, wherein the step of drying further comprises soft baking the liquid coating at about 75° C. for about 1 minute.

12. The method of claim 5, wherein the step of forming a pattern on said surface further comprises the steps of:
a.) exposing a pattern of one or more portions of said photo-reactive layer to electromagnetic radiation having wavelengths within said predetermined range of wavelengths, said photo-absorber absorbing said radiation and, said photo-absorber and said photoacid generator interacting to form an acid species;
b.) heating the coated silicon wafer, wherein said acid species enable an epoxy-epoxide coupling reaction between said short molecule linker epoxide end unit and said epoxy-silane monolayer; and
c.) washing the coated silicon wafer in a polymer solvent and removing the exposed and unexposed portions of said photo-reactive layer providing thereby a plurality of patterned hydrophobic end groups in those areas exposed to said electromagnetic radiation.

13. The method of claim 12, wherein said predetermined range comprises electromagnetic radiation wavelengths between about 300 nm to about 450 nm.

14. The method of claim 12, wherein the step of heating the photoresist coated silicon wafer further comprises the step of baking the coated silicon wafer at about 90° C. for about 3 minutes.

15. A chemical composition for forming a patterning an epoxide surface, comprising glycidyl 4-nonylphenyl ether.

16. The chemical composition of claim 15, further comprising:
a polymer carrier media;
a photoacid generator;
a photo absorber; and
an organic solvent.

17. The chemical composition of claim 16, wherein said polymer carrier media is poly(methyl methacrylate), wherein said poly(methyl methacrylate) has a molecular weight of about 495 kDaltons.

18. The chemical composition of claim 16, wherein said photoacid generator is 4-octyloxyphenyl phenyliodonium hexafluoroantimonate.

19. The chemical composition of claim 16, wherein the photo absorber absorbs light radiation between wavelengths of about 300 nm to about 450 nm.

20. The chemical composition of claim 19, wherein the photo absorber is isopropyl-9H-thioxanthen-9-one.

21. The chemical composition of claim 16, wherein the organic solvent is chlorobenzene.
